(19) United States
(12) Patent Application Publication
(10) Pub. No.: US 2008/0312542 A1
(43) Pub. Date: Dec. 18, 2008

(54) MULTI-SENSOR ARRAY FOR MEASURING BLOOD PRESSURE

(75) Inventors: Matthew J. BANET, Del Mar, CA (US); Henk VISSER, II, San Diego, CA (US); Zhou ZHOU, San Diego, CA (US); Kenneth R. HUNT, Vista, CA (US)

Correspondence Address: WilmerHale/Triage Wireless, 60 State Street, Boston, MA, 02109, US

(73) Assignee: TRIAGE WIRELESS, INC., San Diego, CA (US)

(21) Appl. No.: 12/139,219

(22) Filed: Jun. 13, 2008

Related U.S. Application Data
(60) Provisional application No. 60/943,660, filed on Jun. 13, 2007.

(51) Int. Cl. A61B 5/021

(52) U.S. Cl. 600/485

(57) ABSTRACT

A sensor for monitoring a patient's blood pressure, the sensor including a housing unit with a back surface and which includes: a pair of electrodes mounted on the back surface; an optical system mounted on the back surface and including at least one light source that emits optical radiation near 570 nm and at least one photodetector; a first amplifier which generates an analog electrical waveform from the electrical signals from the electrodes; a second amplifier that generates an analog optical waveform from the optical signal from the photodetector; analog-to-digital converter circuitry configured to receive the analog electrical waveform and generate a digital electrical waveform therefrom and to receive the analog optical waveform and generate a digital optical waveform therefrom; and a processor programmed to receive the digital electrical and optical waveforms and determine a pulse transit time for the patient which is a measure of a separation in time of a first feature of the digital electrical waveform and a second feature of the digital optical waveform and to use the pulse transit time to determine a blood pressure value for the patient.

### CROSS-REFERENCE TO RELATED APPLICATIONS

This application claims the benefit of U.S. Provisional Application No. 60/943,660; filed Jun. 13, 2007, the contents of which are incorporated herein by reference.

### FIELD OF THE INVENTION

The present invention generally relates to medical devices for monitoring vital signs, e.g., blood pressure.

### BACKGROUND OF THE INVENTION

Pulse transit time (PTT), defined as the transit time for a pressure pulse launched by a heartbeat in a patient's arterial system, has been shown in a number of studies to correlate to both systolic and diastolic blood pressures. In these studies, PTT is typically measured with a conventional vital signs monitor that includes separate modules to determine both an electrocardiogram (ECG) and pulse oximetry. During a PTT measurement, multiple electrodes typically attach to a patient's chest to determine a time-dependent ECG component a sharp spike called the ‘QRS complex’. This feature indicates an initial depolarization of ventricles within the heart and, informally, marks the beginning of the heartbeat and pressure pulse that follows. Pulse oximetry is typically measured with a bandage or clothespin-shaped sensor that attaches to a patient's finger, and includes optical systems operating in both the red and infrared spectral regions. A photodetector measures radiation emitted from the optical systems and transmitted through the patient's finger. Other body sites, e.g., the ear, forehead, and nose, can also be used in place of the finger. During a measurement, a microprocessor analyses both red and infrared radiation measured by the photodetector to determine the patient's blood oxygen saturation level and a time-dependent waveform called an optical waveform or photoplethysmograph. Time-dependent features of the optical waveform indicate both pulse rate and a volumetric absorbance change in an underlying artery (e.g., in the finger) caused by the propagating pressure pulse.

Typical PTT measurements determine the time separating a maximum point on the QRS complex (indicating the peak of ventricular depolarization) and a foot of the optical waveform (indicating the beginning the pressure pulse). PTT depends primarily on arterial compliance, the propagation distance of the pressure pulse (closely approximated by the patient's arm length), and blood pressure. PTT typically relates inversely to blood pressure, i.e., a decrease in PTT indicates an increase in blood pressure.

A number of issued U.S. Patents describe the relationship between PTT and blood pressure. For example, U.S. Pat. Nos. 5,316,008; 5,857,975; 5,865,755; and 5,649,543 each describe an apparatus that includes conventional sensors that measure an ECG and optical waveform, which are then processed to determine PTT.

Studies have also shown that a property called vascular transit time (‘VTT’), defined as the time separating two plethysmographs measured from different locations on a patient, can correlate to blood pressure. Alternatively, VTT can be determined from the time separating other time-dependent signals measured from a patient, such as those measured with acoustic or pressure sensors. A study that investigates the correlation between VTT and blood pressure is described, for example, in ‘Evaluation of blood pressure changes using vascular transit time’, Physiol. Meas. 27, 685-694 (2006). U.S. Pat. Nos. 6,511,436; 6,599,251; and 6,723,054 each describe an apparatus that includes a pair of optical or pressure sensors, each sensitive to a propagating pressure pulse, that measure VTT. As described in these patents, a microprocessor associated with the apparatus processes the VTT value to estimate blood pressure.

In order to accurately measure blood pressure, both PTT and VTT measurements typically require a ‘calibration’ measurement consisting of one or more conventional blood pressure measurements made simultaneously with the PTT or VTT measurement. The calibration accounts for patient-to-patient variation in arterial properties (e.g., stiffness and size). Calibration measurements are typically made with an auscultatory technique (e.g., using a pneumatic cuff and stethoscope) at the beginning of the PTT or VTT measurement; these measurements can be repeated if and when the patient undergoes any change that may affect their physiological state. Once completed, the calibration blood pressure measurements are used, along with a change in PTT, to determine the patient's blood pressure and blood pressure variability.

Other efforts have attempted to use a calibration along with other properties of the plethysmograph to measure blood pressure. For example, U.S. Pat. No. 6,616,613 describes a technique wherein a second derivative is taken from a plethysmograph measured from the patient's ear or finger. Properties from the second derivative are then extracted and used with calibration information to estimate the patient's blood pressure. In a related study, described in ‘Assessment of Vasoactive Agents and Vascular Aging by the Second Derivative of Photoplethysmogram Waveform’, Hypertension. 32, 365-370 (1998), the second derivative of the plethysmograph is analyzed to estimate the patient's ‘vascular age’ which is related to the patient's biological age and vascular properties.

### SUMMARY OF THE INVENTION

This invention provides a sensor armband that includes an embedded multi-sensor array and electrode that, together, make a cuffless measurement of blood pressure using PTT. Once measured, the PTT value may be corrected by a property, referred to herein as a ‘vascular index’ (‘VI’), that accounts for the patient's arterial properties (e.g., stiffness and size). VI is typically determined by the shape of the plethysmograph, referred to herein as an ‘optical waveform’, which is measured from the brachial, finger, radial, or ulnar arteries. To accurately measure VI, the optical waveform must be characterized by a high signal strength and signal-to-noise ratio. The multi-sensor array according to the invention measures such a signal because it includes multiple (e.g. three or more) optical modules, wired together and collectively working in concert, to measure a single signal.

The invention has a number of advantages. In general, the armband described herein uses both PTT and VI to make a cuffless measurement of blood pressure without requiring calibration at the beginning of the measurement. This dramatically simplifies the process of measuring blood pressure on a patient in the hospital and home setting. Moreover, the armband can send information through either a wired or wireless interface to an external device that combines all the data-analysis features and form factor of a conventional PDA. The wireless interface, in particular, increases patient mobility by eliminating the wires that normally tether a patient to a conventional vital signs monitor. Ultimately this results in an easy-to-use, flexible system that performs one-time, continuous, and ambulatory measurements both in and outside of a hospital. Measurements can be made throughout the day with little or no inconvenience to the caregiver or patient.

These and other advantages are described in detail in the following description, and in the claims.

### BRIEF DESCRIPTION OF THE DRAWINGS

FIG. 1 is a schematic view of a sensor armband, attached to a patient, including a multi-sensor array and electrode;

FIG. 2 is a schematic view of the multi-sensor array of FIG. 1 including three optical modules;

FIG. 3 is a schematic view of the three optical modules of FIG. 2 measuring a patient through several layers of epidermis;

FIG. 4 is a schematic view of the sensor armband of FIG. 1 featuring electrodes and a multi-sensor array;

FIG. 5 is a schematic of the front and side views, respectively, of a circuit board and battery housed within the sensor armband of FIG. 4.

### DETAILED DESCRIPTION

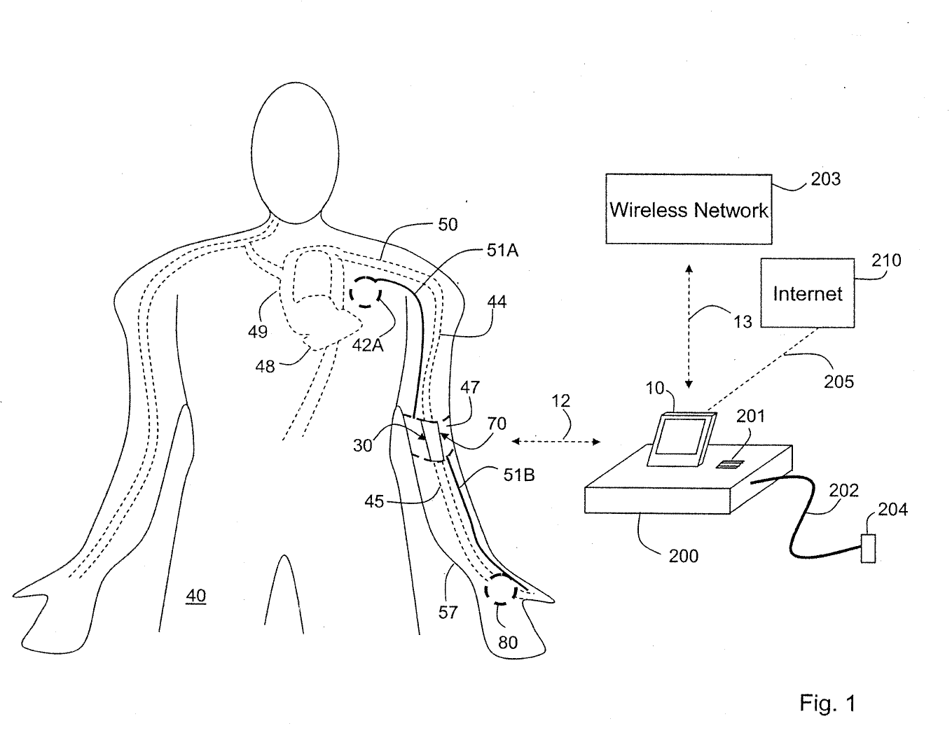

FIG. 1 shows a sensor armband 47 according to the invention featuring a multi-sensor array 30 that measures blood pressure from a patient 40. During a measurement, the patient's heart 48 generates electrical impulses that pass through the body near the speed of light. These impulses stimulate each heart beat, which in turn generates a pressure wave that propagates through the patient's vasculature at a significantly slower speed. Immediately after the heartbeat, the pressure wave leaves the aorta 49, passes through the subclavian artery 50, to the brachial artery 44, and from there through the radial artery 45 to smaller arteries in the patient's fingers. The armband 47 includes an embedded two-part electrode 70 and connects to a third electrode 42A, attached to the patient's chest, through a cable 51A. Collectively, these three electrodes 70, 42A measure unique electrical signals which pass to an amplifier/filter circuit within an embedded electronics module. The amplifier/filter circuits are conventional circuits that include analog band-pass filters that typically pass frequencies between 1 and 50 Hz, and conventional amplifiers with fixed or adjustable (e.g. software-controlled) gain. These circuits process the signals to generate an analog electrical signal, similar to a conventional ECG, which is then digitized with an analog-to-digital converter to form the electrical waveform that is stored in memory.

Using a reflection-mode geometry, the multi-sensor array 30 embedded in the sensor armband 47 measures an optical waveform from the patient's brachial artery. A second optical sensor 80 connected to the electronics module through a cable 51B can additionally measure a second optical waveform from the patient's radial or ulnar artery; typically the second optical sensor 80 is disposed on the underside of the patient's wrist 57. These signals are amplified using second and third amplifier/filter circuits, and then digitized with second and third channels within the analog-to-digital converter in the electronics module. Each optical waveform features a time-dependent ‘pulse’ corresponding to each heartbeat that represents a volumetric change in an underlying artery caused by the propagating pressure pulse. The electrical waveform includes a sharp peak corresponding to the QRS complex. PTT is calculated for each heartbeat by measuring the time difference between the peak of the electrical waveform and the foot of at least one optical waveform. An algorithm processes PTT to determine the patient's blood pressure. As described above, PTT and blood pressure typically relate through an inverse, linear relationship.

The above-described system can be used in a number of different settings, including both the home and hospital. A patient 40 in a hospital, for example, can continuously wear the sensor armband 47 over a time period ranging from minutes to several days. During this period, the sensor armband 47 is powered by a rechargeable battery, and continuously measures blood pressure along with other vital signs. At a predetermined interval (typically, e.g., every few minutes) the sensor armband transmits this information through a short-range wireless interface 12 (e.g., a Bluetooth® interface) to the bedside device 10, which is typically seated in a docking station 200 next to a bed in the hospital. The docking station 200 allows the device 10 to be easily seen by the patient or caregiver and additionally includes an AC adaptor 202 that plugs into a wall outlet 204 and continuously charges the device's battery as well as a spare battery 201 for the armband 47. When the original rechargeable battery in the armband is depleted, the caregiver (or patient) 40 replaces it with the spare battery 201 in the docking station 200. The device 10 is highly portable and can be easily removed from the docking station 200. It communicates with a nation-wide wireless network (e.g. Sprint) through a long-range wireless interface 13 (e.g., a CDMA modem), or with the Internet 210 through a wired or wireless (e.g., 802.11) interface 205.

Each optical module within the multi-sensor array 30 typically includes an LED operating near 570 nm, a photodetector, and an amplifier. This wavelength is selected because it is particularly sensitive to volumetric changes in an underlying artery when deployed in a reflection-mode geometry, as described in the following co-pending patent application, the entire contents of which are incorporated herein by reference: SYSTEM FOR MEASURING VITAL SIGNS USING AN OPTICAL MODULE FEATURING A GREEN LIGHT SOURCE (U.S. Ser. No. 11/307,375; filed Feb. 3, 2006). 570 nm is also particularly effective at measuring optical waveforms from a wide range of skin types featuring different levels of pigmentation. Use of this wavelength is described, for example, in the following technical paper, the contents of which are incorporated herein by reference: ‘Racial Differences in Aortic Stiffness in Normotensive and Hypertensive Adults’, Journal of Hypertension. 17, 631-637, (1999). A preferred optical module is the TRS1755 manufactured by TAOS Inc. of Plano, Tex. (www.taosinc.com). In other embodiments, the integrated optical module can be replaced by one or more stand-alone photodetectors and LEDs operating near 570 nm.

Typically, three optical modules are used in the multi-sensor array 30 to increase the effective area they irradiate and, consequently, the probability that an underlying or proximal artery is measured. This in turn increases both the strength of the optical signal and its signal-to-noise ratio. Operating in concert, the three sensors collectively measure an optical waveform that includes photocurrent generated by each optical module. The resultant signal forms the optical waveform, and effectively represents an additive or summation signal measured from vasculature (e.g., arteries and capillaries) underneath or proximal to the sensor 30. The secondary sensor 80 typically includes a similar optical module, and can additionally include LEDs operating near 650 nm and 950 nm to make a pulse oximetry measurement.

The above-described system determines the patient's blood pressure using PTT, and then corrects this value for VI using the algorithm described below. Specifically, it is well know that a patient's arteries stiffen with biological age. This property can thus be used to estimate the patient's vascular stiffness. When used with a PTT-based measurement of blood pressure, which depends strongly on vascular stiffness, biological age can therefore reduce the need for calibration and increase the accuracy of the blood pressure measurement. The accuracy of the measurement can be further improved with VI, which serves as a proxy for a ‘true’ age of the patient's vasculature: patients with elastic arteries for their age will have a VI lower than their biological age, while patients with stiff arteries for their age will have a VI greater than their biological age. The difference between VI and the patient's biological age can be compared to a pre-determined correction factor to improve the accuracy of a PTT-based blood pressure measurement.

Co-pending patent applications that describe methods for calculating VI and using it in a PTT-based measurement are described below. Their entire contents are hereby incorporated by reference: 1) VITAL SIGN MONITOR FOR CUFFLESSLY MEASURING BLOOD PRESSURE WITHOUT USING AN EXTERNAL CALIBRATION (U.S. Ser. No. 11/682,228; filed Mar. 5, 2007); 2) VITAL SIGN MONITOR FOR MEASURING BLOOD PRESSURE USING OPTICAL, ELECTRICAL, AND PRESSURE WAVEFORMS (U.S. Ser. No. 12/138,194, filed Jun. 12, 2008); and, 3) VITAL SIGN MONITOR FOR CUFFLESSLY MEASURING BLOOD PRESSURE CORRECTED FOR VASCULAR INDEX (U.S. Ser. No. 12/138,199, filed Jun. 12, 2008).

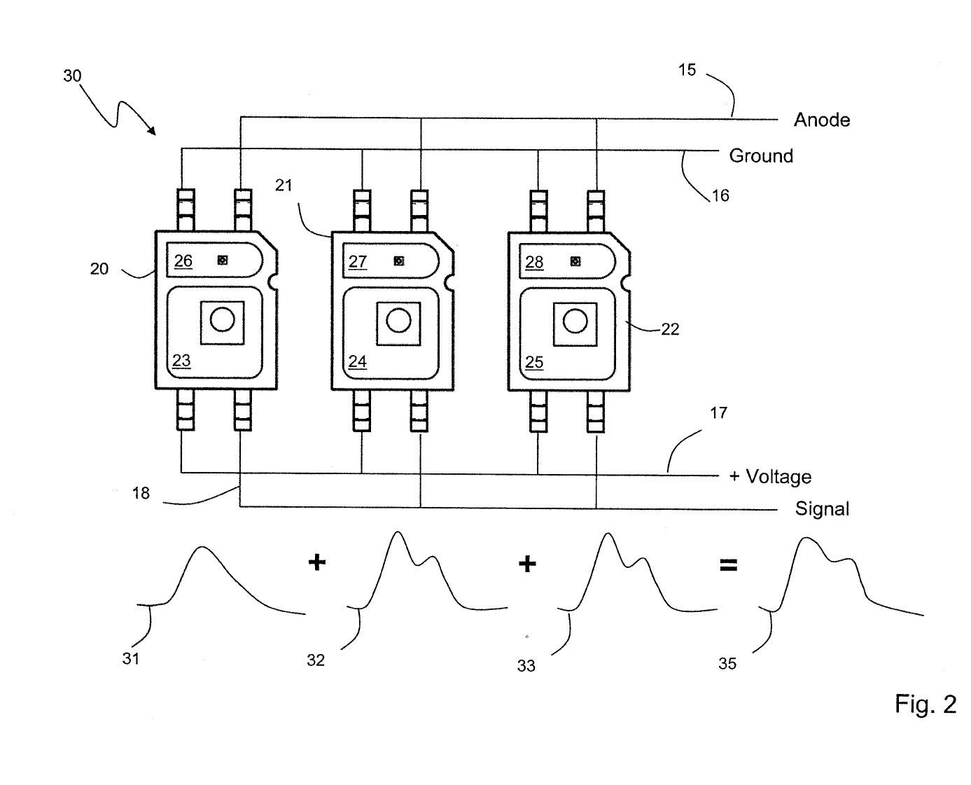

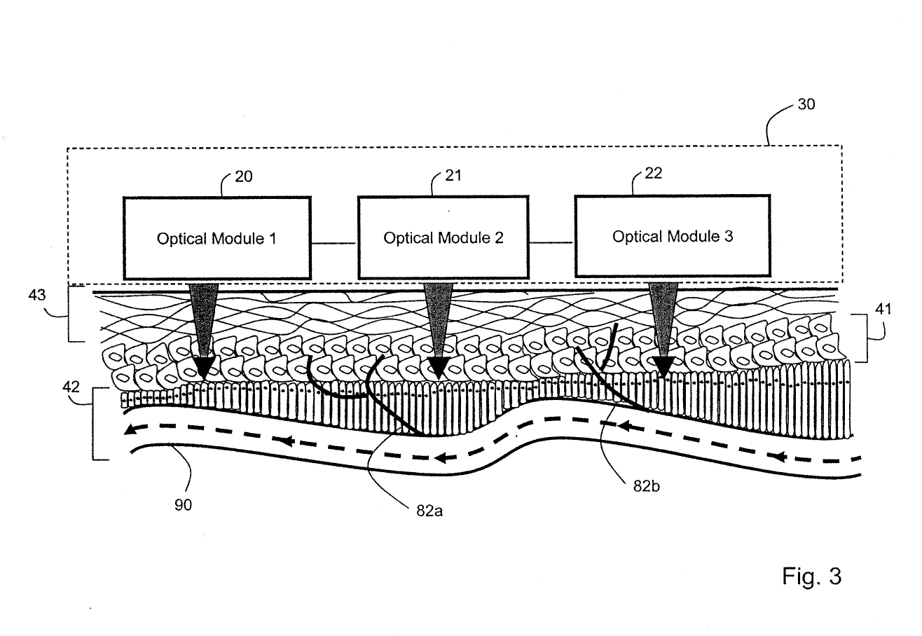

Referring to FIGS. 2 and 3, the multi-sensor array 30 operates collectively using three optical modules 20, 21, 22, each containing an LED 26, 27, 28 (typically operating near 570+/−10 nm), a photodetector 23, 24, 25, and a built-in amplifier (not shown in the figure). The modules 20, 21, 22 are wired together in parallel using 4 pins total: +Voltage 17, Ground 16, Anode 15, and Signal 18. With this wiring configuration the modules 20, 21, 22 are powered and simultaneously measure a signal from an underlying or proximal artery. Each LED 26, 27, and 28 generates radiation near 570 nm, which passes through the epidermis 43, dermis 41, and subcutis 42 to irradiate blood flowing in the underlying artery 90 and capillaries 82a, 82b. As the heartbeat-induced pressure pulse passes through these flexible vessels, it increases an internal bolus of blood that causes the vessels to temporarily increase in diameter. This, in turn, increases the amount of radiation absorbed according to Beer's Law, and decreases the amount of reflected radiation that irradiates each of the three photodetectors 23, 24, 25. In response to the incident light, each the three photodetectors 23, 24, 25 generate photocurrent that is amplified by the built-in amplifier. Each photodetector 23, 24, 25 may collect reflected light that originates from an LED 26, 27, 28 contained in any of the optical modules 20, 21, 22. Preferably the modules 20, 21, 22 are spaced within 1-2 mm so that this occurs. Once light is collected by the photodetector 23, 24, 25, the built-in amplifier in each optical module amplifies the resultant photocurrent to generate a unique optical waveform 31, 32, and 33 (note: the waveforms shown in FIG. 2 increase in intensity with each heartbeat, and thus represent the inverse of the signal measured at the photodetector). Photocurrent representing each waveform 31, 32, 33 merges within the signal 18 line to form a collective signal 35 that then passes to the amplifier/filter circuit within the armband's electronics module for further processing. This yields a filtered, digital optical waveform, which is then processed as described above for the PTT measurement of blood pressure.

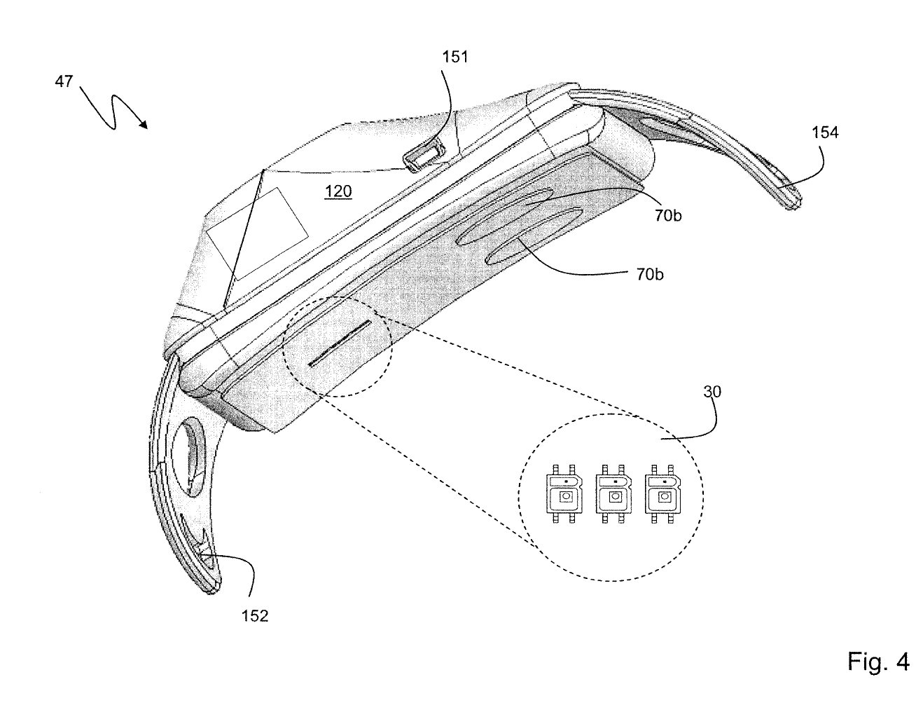

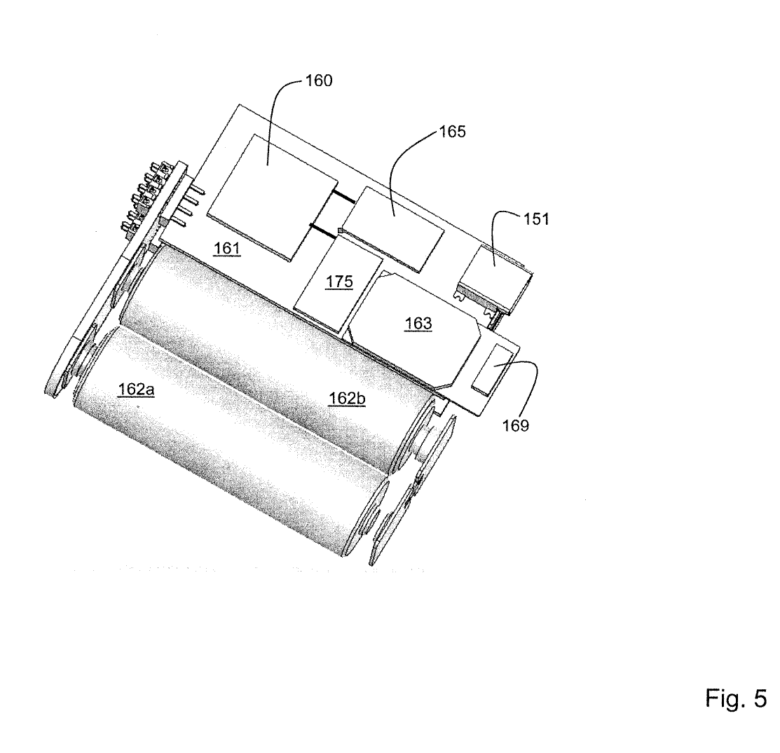

As shown in FIGS. 4 and 5, the armband 47 features a low-profile housing 120 that includes electrodes 70a, 70b and the multi-sensor array 30. The housing 120 is typically made of a flexible rubber or plastic and may be either disposable or non-disposable. During a measurement, the armband 47 is strapped to a patient's arm using a flexible strap (not shown in the figure) that connects to molded D-ring connectors 152, 154 on each side of the housing 120. In this configuration the multi-sensor array 30 and electrodes 70a, 70a contact the patient's skin to measure the optical and electrical waveforms as described above. Note that multi-sensor array 30 and electrodes 70a, 70a are all arranged on a surface of the housing that is held up against the patient's arm when the armband is strapped to the patient's arm.

A main circuit board 161, powered by two rechargeable AA batteries 162a, 162b, supports surface-mounted electronic components within the housing 30. Computer code that controls the armband's various functions and algorithms runs on a high-end microprocessor 160, typically an ARM 9 processor (manufacturer: Atmel; part number: AT91SAM9261-CJ) contained in a ‘ball grid array’ package. Before being processed by the microprocessor 160, analog signals from the multi-sensor array 30 and electrodes 70a, 70b pass to an analog-to-digital converter 165, which is typically a separate integrated circuit (manufacturer: Texas Instruments; part number: ADS8344NB) that digitizes the waveforms at 1 KHz with 16-bit resolution. Such high resolution is required to adequately process the optical and electrical waveforms and generate an accurate PTT value. Once digitized, the waveforms can be stored in memory 175 external to the memory in the microprocessor 160 for further processing.

The armband 47 additionally includes an embedded, short-range wireless Bluetooth® transceiver 163 to wirelessly transmit blood pressure and other information to an external device through an on-board ceramic antenna 169 (manufacturer: BlueRadios; part number: BR-C40A). The Bluetooth® transceiver 163 can be replaced with an alternative wireless transceiver that operates on a wireless local-area network, such as a WiFi® transceiver (manufacturer: DPAC; part number: WLNB-AN-DP101). Wired connections to, e.g., computers are made with a standard mini-USB connection 151.

A number of additional approaches can be used to calculate blood pressure from PTT measured as described above. Such method are described in the following co-pending patent applications, the contents of which are incorporated herein by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No. filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) PATCH SENSOR FOR MEASURING VITAL SIGNS (U.S. Ser. No. 11/160,957; filed Jul. 18, 2005); 10) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 11) HAND-HELD MONITOR FOR MEASURING VITAL SIGNS (U.S. Ser. No. 11/162,742; filed Sep. 21, 2005); 12) CHEST STRAP FOR MEASURING VITAL SIGNS (U.S. Ser. No. 11/306,243; filed Dec. 20, 2005); 13) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 14) BILATERAL DEVICE, SYSTEM AND METHOD FOR MONITORING VITAL SIGNS (U.S. Ser. No. 11/420,281; filed May 25, 2006); 15) SYSTEM FOR MEASURING VITAL SIGNS USING BILATERAL PULSE TRANSIT TIME (U.S. Ser. No. 11/420,652; filed May 26, 2006); 16) BLOOD PRESSURE MONITOR (U.S. Ser. No. 11/530,076; filed Sep. 8, 2006); 17) TWO-PART PATCH SENSOR FOR MONITORING VITAL SIGNS (U.S. Ser. No. 11/558,538; filed Nov. 10, 2006); and, 18) MONITOR FOR MEASURING VITAL SIGNS AND RENDERING VIDEO IMAGES (U.S. Ser. No. 11/682,177; filed Mar. 5, 2007).

Other embodiments are also within the scope of the invention. For example, the system is not limited to three optical modules. Additional optical modules could be added to further strengthen the magnitude of the optical waveform. Also, the optical modules within the multi-sensor array are not limited to the ‘linear’ form factor shown in FIG. 2. The modules, for example, may be placed in a circular configuration, may be offset from one another, or may be fashioned in a random distribution to irradiate a relatively large area of underlying skin. Such a configuration may be desirable for patients with darker pigment. In other embodiments, additional electrodes may be added to strengthen the electrical waveform.

Still other embodiments are covered by the following claims.

What is claimed is:

1. A sensor for monitoring a patient's blood pressure, the sensor comprising a housing unit configured to be worn on a patient's arm, the housing unit having a back surface and comprising:
a pair of electrodes mounted on said back surface and positioned to contact the patient's skin to generate electrical signals when the housing is worn on the patient's arm;
an optical system mounted on said back surface and comprising at least one light source that emits optical radiation near 570 nm and at least one photodetector, wherein the at least one light source and the at least one photodetector are positioned to be adjacent to the patient's skin when the housing is worn on the patient's arm, wherein optical system is configured to generate an optical signal by detecting optical radiation emitted by the at least one light source and reflected from blood vessels underneath the patient's skin;
a first amplifier configured to receive the electrical signals from the electrodes and generate an analog electrical waveform therefrom;
a second amplifier configured to receive the optical signal from the photodetector and generate an analog optical waveform therefrom;
analog-to-digital converter circuitry configured to receive the analog electrical waveform and generate a digital electrical waveform therefrom and to receive the analog optical waveform and generate a digital optical waveform therefrom; and
a processor programmed to receive the digital electrical and optical waveforms and determine a pulse transit time for the patient which is a measure of a separation in time of a first feature of the digital electrical waveform and a second feature of the digital optical waveform and to use the pulse transit time to determine a blood pressure value for the patient.

2. The sensor of claim 1, further comprising a short-range wireless transceiver configured to receive the blood pressure value for the patient and transmit it to a remote receiver.

3. The sensor of claim 1, further comprising an armband attached to the housing unit and for attaching the housing unit to the patient's arm.

4. The sensor of claim 1, wherein the optical system comprises an array of optical modules mounted on said back surface and positioned to be adjacent to the patient's skin when the housing is worn on the patient's arm, wherein each optical module of the array of optical modules comprises a light source that emits optical radiation near 570 nm and a photodetector configured to generate an optical signal by detecting optical radiation emitted by the light source within that optical module and reflected from blood vessels underneath the patient's skin and wherein the at least one light source and the at least one photodetector are among the light sources and photodetectors of the array.

5. The sensor of claim 1, wherein the optical modules are linearly arranged on said back surface.

6. The sensor of claim 1, wherein the optical modules are arranged in a circular pattern on said back surface.

7. A sensor for monitoring a patient's blood pressure, the sensor comprising a housing unit configured to be worn on a patient's arm, the housing comprising:
a pair of electrodes mounted on said back surface and positioned to contact the patient's skin to generate electrical signals when the housing is worn on the patient's arm;
an array of optical modules mounted on said back surface and positioned to be adjacent to the patient's skin when the housing is worn on the patient's arm, wherein each optical module of the array of optical modules comprises a light source that emits optical radiation near 570 nm and a photodetector configured to generate an optical signal by detecting optical radiation emitted by the light source within that optical module and reflected from blood vessels underneath the patient's skin, wherein each optical module outputs on an output line an output signal that is derived from the optical signal in that optical module, wherein the output lines of the plurality of optical modules are all electrically connected to a common signal line to thereby produce a collective optical signal on the common signal line;
a first amplifier configured to receive the electrical signals from the electrodes and generate an analog electrical waveform therefrom;
a second amplifier configured to receive the collective optical signal from the collective signal line and generate an analog optical waveform therefrom;
analog-to-digital converter circuitry configured to receive the analog electrical waveform and generate a digital electrical waveform therefrom, and configured to receive the analog optical waveform and generate a digital optical waveform therefrom; and
a processor programmed to receive the digital electrical and optical waveforms and determine a pulse transit time for the patient which is a measure of a separation in time of a first feature of the digital electrical waveform and a second feature of the digital photodetector waveform and to use the pulse transit time to determine a blood pressure value for the patient.

8. The sensor of claim 7, wherein the collective optical signal is a sum of the output signals from the array of optical modules.

9. The sensor of claim 7, wherein the optical modules are linearly arranged on said back surface.

10. The sensor of claim 7, wherein the optical modules are arranged in a circular pattern on said back surface.

11. The sensor of claim 7, further comprising a short-range wireless transceiver configured to receive the blood pressure value for the patient and transmit it to a remote receiver.

12. The sensor of claim 7, further comprising an armband attached to the housing unit and for attaching the housing unit to the patient's arm.
